(19) United States
(12) Patent Application Publication
(10) Pub. No.: US 2002/0156452 A1
(43) Pub. Date: Oct. 24, 2002

(54) Method and apparatus for curving catheter with soft distal end

(76) Inventors: Matt D. Pursley, Alpharetta, GA (US); Joe E. Brown, Lilburn, GA (US)

Correspondence Address: THOMPSON & THOMPSON, P.A., 310 4th Street, P.O. Box 66, Scandia, KS, 66966, US

(21) Appl. No.: 10/167,718

(22) Filed: Jun. 11, 2002

Related U.S. Application Data
(63) Continuation-in-part of application No. 09/558,078, filed on Apr. 25, 2000, now Pat. No. 6,402,736.
(63) Continuation-in-part of application No. 09/168,133, filed on Oct. 7, 1998, now Pat. No. 6,053,900.
(63) Continuation-in-part of application No. 08/801,576, filed on Feb. 14, 1997, now abandoned.
(63) Continuation-in-part of application No. 08/602,424, filed on Feb. 16, 1996, now Pat. No. 5,603,694.
(60) Provisional application No. 60/298,134, filed on Jun. 13, 2001.
(60) Provisional application No. 60/088,152, filed on Jun. 5, 1998.

(51) Int. Cl. A61M 31/00

(52) U.S. Cl. 604/500; 604/530

(57) ABSTRACT

An apparatus and method for curving a catheter include a resilient fiber core with a preformed curve shape and a catheter having a lumen. The resilient fiber core is inserted into the lumen to create a desired curve shape in the catheter. The lumen in which the fiber core is inserted can be the primary lumen of the catheter, a secondary lumen, or a channel in the wall of the catheter. The catheter can be formed of a soft polymer material. The fiber core can be inserted into the catheter before or after the catheter is introduced into a body. The method can be used with a catheter having a varying hardness over its length, including a soft portion that conforms to the curve shape of the fiber core. A positive or negative pressure can be applied to a channel in the catheter to selectively straighten or curve the catheter.

### RELATED APPLICATIONS

This application claims the benefit of Applicants' Provisional Patent Application No. 60/298,134 filed on Jun. 13, 2001, and is a continuation-in-part of Applicants' copending U.S. application Ser. No. 09/558,078, filed on Apr. 25, 2000, now U.S. Pat. No. 6,402,736, which is a continuation-in-part of Applicants' copending U.S. application Ser. No. 09/168,133, filed on Oct. 7, 1998, now U.S. Pat. No. 6,053,900, which claims the benefit of U.S. Provisional Application Serial No. 60/088,152 filed on Jun. 5, 1998, and is a continuation-in-part of Applicants' copending International Application No. PCT/US98/02770 filed Feb. 13, 1998, which is a continuation-in-part of Applicants' copending U.S. application Ser. No. 08/801,576, filed on Feb. 14, 1997, now abandoned, which is a continuation-in-part of Applicants' copending U.S. application Ser. No. 08/602,424, filed Feb. 16, 1996, now U.S. Pat. No. 5,603,694. This application is also related to the Applicants' U.S. application Ser. No. 08/918,713, filed on Aug. 22, 1997, now U.S. Pat. No. 6,030,371. The contents of these prior applications and patents are incorporated herein by reference.

### BACKGROUND OF THE INVENTION

1. Field of the Invention

The present invention relates generally to catheters and, in particular, to catheters having curved shapes and methods for making and deploying catheters having curved shapes.

### DESCRIPTION OF THE RELATED ART

Catheters frequently have “preset” curves in them to enhance the physician's ability to introduce the catheter to the desired location. Usually, this curve is “set” in the catheter by first bending the catheter to the desired shape, then applying heat to the catheter while in this curved state, and then allowing the catheter to cool while still in this curved shape. The plastic memory of the polymer allows the curved shape to be maintained after cooling. In some cases, the catheter can be curved without heat by cold working the catheter into a curved shape.

The existing methods of curving catheters have two main disadvantages. First, the curved shape requires some rigidity of the catheter to maintain the curve shape. Second, the curve can only be “set” outside of the body.

Thus, there is a need in the industry for an improved method and apparatus for making and deploying catheters having a curved shape.

### SUMMARY OF THE INVENTION

It is an object of the present invention to provide a method and apparatus for curving a catheter having a soft distal end that overcomes the problems in the above-mentioned prior art.

It is a further object of the present invention to provide an improved method for curving a catheter having a very soft polymer.

It is a further object of the present invention to provide an improved method for introducing curves into catheters while the catheter remains inside the body.

To achieve the foregoing and other objects and in accordance with the purpose of the present invention, as embodied and broadly described herein, the method for curving a catheter according to the present invention comprises: providing a resilient fiber core with a preformed curve shape; providing a catheter having a lumen; and inserting the resilient fiber core into the lumen of the catheter to create a desired curve shape in the catheter. The catheter is preferably formed of a soft polymer tubing, and the resilient fiber core can be inserted into the catheter before or after the catheter is introduced into a body.

The catheter can also have a varying hardness over its length with a relatively hard portion and a relatively soft portion. In this case, the resilient fiber core conforms to a shape of the relatively hard portion of the catheter, and the relatively soft portion of the catheter conforms to the preformed curve shape of the resilient fiber core.

According to other embodiments of the invention, a positive or negative pressure can be applied to a channel in the catheter to selectively straighten or curve the catheter.

According to another embodiment of the invention, the catheter has a plurality of lumens, and a plurality of resilient fiber cores having preformed curve shapes are inserted into the respective lumens to create a desired curve shape in the catheter.

Additional objects, advantages, and novel features of the invention will be set forth in the following description, and will become apparent to those skilled in the art upon reading this description or practicing the invention. The objects and advantages of the invention may be realized and attained by the appended claims.

### BRIEF DESCRIPTION OF THE DRAWINGS

The present invention will become more clearly appreciated as the disclosure of the present invention is made with reference to the accompanying drawings. In the drawings:

FIG. 1 is a perspective view of a catheter which is curved using the technique of the present invention.

FIG. 2 is an enlarged perspective view of the curved catheter shown in FIG. 1.

FIG. 3 is a cross section view of a catheter having a varying hardness along its length and a resilient fiber core placed in a channel in the wall of the catheter.

FIG. 4 is a section view of a catheter having multiple lumens in which a plurality of resilient fiber cores are inserted to curve the catheter.

FIG. 5 is a section view of a catheter having a first lumen for receiving a resilient fiber core, a second lumen for delivering a fluid-based agent, and a third lumen which can be pressurized or depressurized to straighten or curve the catheter.

### DETAILED DESCRIPTION OF THE INVENTION

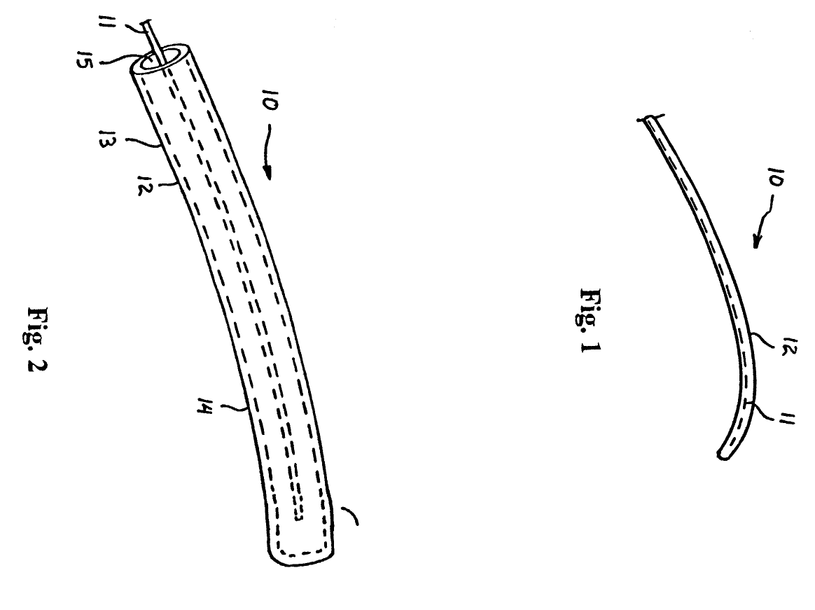

Methods and apparatus for curving a catheter according to the present invention will be described in detail hereinafter with reference to FIGS. 1 to 5 of the accompanying drawings.

A curved catheter 10 according to a first embodiment of the present invention is shown in FIG. 1. The curved catheter 10 includes a small diameter, resilient fiber core 11, and a soft polymer tubing 12 that encases the resilient fiber core 11 and adapts to the shape of the fiber core 11. The fiber core 11 has a preformed curve shape which corresponds to the desired curve shape of the catheter 10. The soft polymer tubing 12 conforms to the curve shape of the fiber core 11, thereby creating a catheter 10 with a desired curve shape.

The fiber core 11 can be formed of a metallic wire, boron fiber or other suitable resilient material. The polymer tubing 12 can be formed of a nylon, urethane, PE, TFE, or other suitable polymer material which is very soft and offers virtually no resistance to the preformed shape of the resilient fiber core 11.

The polymer tubing 12 can be formed, for example, using the nonextrusion manufacturing method and apparatus described in Applicants' U.S. Pat. No. 6,030,371. Using this method, the polymer tubing 12 can be formed with a variable hardness over the length of the catheter by continuously changing the constituents or mixtures of the polymer material(s) being used. The catheter can thus have a relatively stiff or hard portion 13 near its proximal end and a relatively softer portion 14 near its distal end. The soft portion 14 of the catheter 10 conforms to the preformed curve shape of the fiber core 11 when the fiber core 11 is inserted into the catheter 10, while the hard portion 13 remains straight or in its preset shape. A gradual change in hardness can be provided between the hard portion 13 and the softer portion 14 using the nonextrusion manufacturing method and apparatus of the '371 patent.

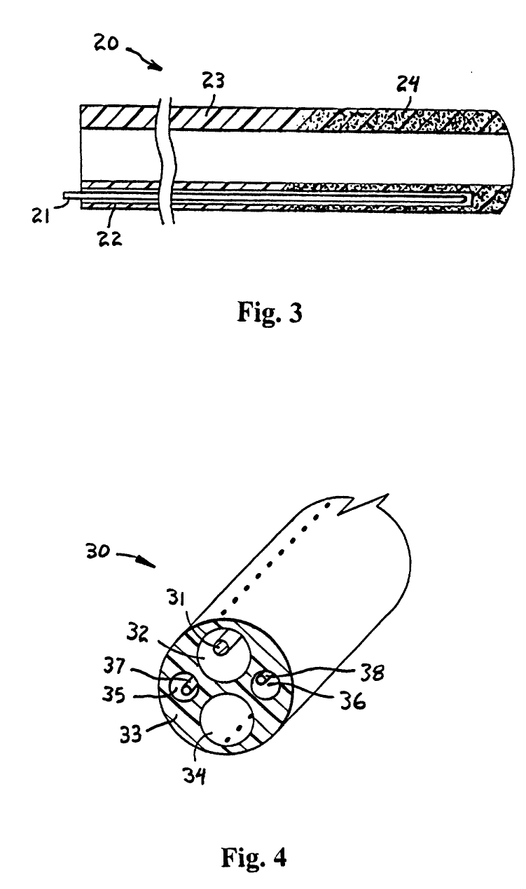

The resilient fiber core 11 is preformed to the desired curve shape and then introduced into a soft section of the polymer tubing 12. The fiber core 11 can be introduced into the polymer tubing 12 through the primary lumen 15 of the catheter 10, as shown in FIG. 2. In an alternative embodiment shown in FIG. 3, a catheter 20 is provided in which the fiber core 21 is inserted into a lumen or channel 22 in the wall of the catheter 20. In another alternative embodiment shown in FIG. 4, a catheter 30 is provided in which the fiber core 31 is inserted into a secondary lumen 32 leaving the primary lumen 34 open.

The resilient fiber core 11, 21, 31 can be placed into the catheter 10, 20, 30 in each of the above-described embodiments prior to its usage to form a catheter with a precurved shape. Alternatively, the catheter 10, 20, 30 can be inserted into the body and the curved fiber core 11, 21, 31 introduced into the catheter while the catheter is within the body. This method will work particularly well with a variable hardness catheter 20, such as that shown in FIG. 3 and produced by the nonextrusion manufacturing method of U.S. Pat. No. 6,030,371. The resilient fiber core 21 will remain straight while it transits the hard portion 23 of the catheter 30. The resilient fiber core 21 will then bend the catheter 20 into the desired shape as it makes its way into the softer portion 24 of the catheter 20.

A method for curving a catheter 30 according to another embodiment of the present invention will now be explained with reference to FIG. 4. In this embodiment, a polymer tubing 33 for the catheter 30 is formed with multiple lumens that run the length of the catheter 30. For example, four lumens 32, 34, 35, 36 can be formed in the catheter 30 at circumferentially spaced positions. Resilient fiber cores 31, 37, 38 having preformed curve shapes can then be inserted into a plurality of the lumens 32, 35, 36 to create a desired curve shape in the catheter 30. In this embodiment, the amount of curvature in the catheter 30 can be controlled or changed during a medical procedure by increasing or decreasing the number of fiber cores 31, 37, 38 inserted into the respective lumens 32, 35, 36. The other lumen 34 can be used for delivering fluid-based agents through the catheter 30. The polymer tubing 33 with multiple lumens 32, 34, 35, 36 can be formed using the nonextrusion manufacturing method described in Applicants' U.S. Pat. No. 6,030,371.

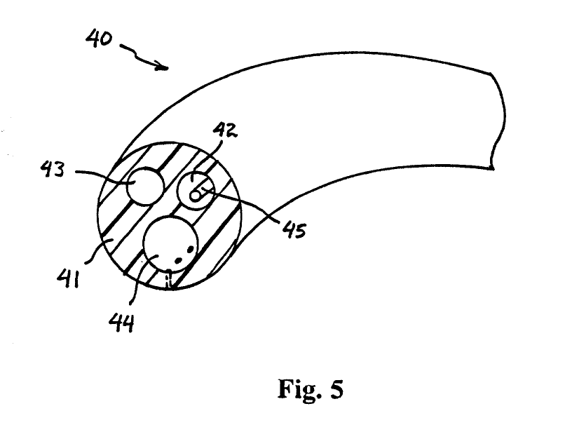

A method of curving a catheter 40 according to an alternative embodiment of the present invention will now be described with reference to FIG. 5. In this embodiment, a polymer tubing 41 for the catheter 40 having multiple lumens or channels 42, 43, 44 is produced using, for example, the nonextrusion method of manufacturing described in Applicants' U.S. Pat. No. 6,030,371. A resilient fiber core 45 having a precurved shape is inserted into a first lumen 42 of the polymer tubing 41 of the catheter 40 prior to its use. A second lumen or channel 43 in the wall of the catheter 40 is then pressurized to straighten the catheter 40. The straightened catheter 40 is then inserted into the body to its desired destination. As the catheter 40 reaches its destination, the pressure on the channel 43 is partially or completely removed so that the curve in the precurved shape of the resilient fiber core 45 can take its shape again. A third lumen 44 is provided for delivering fluid-based agents through the catheter 40.

This alternative method can be useful in neurovascular applications where it is preferable to insert a straight catheter to the site and then introduce a curve after the catheter reaches the site. The tortuous path of the catheter would produce too much friction for movement of the resilient fiber core to form the curve using a deflection wire or to slide a precurved wire into the catheter.

Although the catheter 40 in this embodiment has three separate lumens 42, 43, 44 for introducing the fiber core 45, introducing a pressure, and delivering fluid-based agents, respectively, it is contemplated that these functions can be accomplished with fewer lumens. For example, a pressure for straightening the catheter 40 can be introduced into the same lumen 42 as the fiber core 45 is introduced.

A method for curving a catheter 40 according to another embodiment of the present invention will now be explained with reference again to FIG. 5. The polymer tubing 41 is formed with multiple lumens or channels 42, 43, 44 as explained above. The fiber core 45 is preformed with a generally straight shape or a slightly curved shape. The polymer tubing 41 conforms to the straight or slightly curved shape of the fiber core 45, and the catheter 40 is inserted to the desired site as a straight or slightly curved catheter. After it reaches the site, a negative pressure is applied to the second lumen or channel 43 to cause a more substantial curve to form in the catheter 40. The curve is maintained in the catheter 40 until the negative pressure is removed, at which point the catheter 40 becomes straight or only slightly curved again and can be removed easily.

While the invention has been specifically described in connection with specific embodiments thereof, it is to be understood that this is by way of illustration and not of limitation, and the scope of the appended claims should be construed as broadly as the prior art will permit.

What is claimed is:

1. A method for curving a catheter, comprising:
providing a resilient fiber core with a preformed curve shape;
providing a catheter having a lumen; and
inserting the resilient fiber core into the lumen of the catheter to create a desired curve shape in the catheter.

2. The method according to claim 1, wherein the catheter is formed of a soft polymer tubing that conforms to the preformed curve shape of the resilient fiber core.

3. The method according to claim 1, wherein the fiber core is inserted into the catheter before the catheter is introduced into a body.

4. The method according to claim 1, wherein the fiber core is inserted into the catheter after the catheter is introduced into a body.

5. The method according to claim 1, wherein the catheter has a varying hardness over its length with a relatively hard portion and a relatively soft portion, the fiber core conforms to a shape of the relatively hard portion of the catheter, and the relatively soft portion of the catheter conforms to the preformed curve shape of the fiber core.

6. The method according to claim 1, further comprising the step of selectively applying a pressure to a lumen in the catheter to straighten the catheter.

7. The method according to claim 1, further comprising the step of selectively applying a negative pressure to a lumen in the catheter to change a curve in the catheter.

8. The method according to claim 1, wherein the catheter has a plurality of lumens, and a plurality of resilient fiber cores having preformed curve shapes are inserted into respective lumens to create a desired curve shape in the catheter.

9. A method for deploying a catheter, comprising:
providing a resilient fiber core having a preformed shape;
providing a catheter having a lumen for receiving the resilient fiber core, said catheter having a soft portion of its length that conforms to the preformed shape of the resilient fiber core; and
causing the soft portion of the catheter to conform to the preformed shape of the resilient fiber core during deployment.

10. The method according to claim 9, wherein the soft portion of the catheter is formed of a soft polymer tubing.

11. The method according to claim 9, wherein the resilient fiber core is inserted into the catheter before the catheter is introduced into the patient's body.

12. The method according to claim 9, wherein the resilient fiber core is inserted into the catheter after the catheter is introduced into the patient's body.

13. The method according to claim 9, wherein the catheter has a varying hardness over its length with a relatively hard portion that does not conform to the preformed shape of the resilient fiber core.

14. The method according to claim 9, further comprising the step of selectively applying a pressure to a lumen in the catheter to straighten the catheter.

15. The method according to claim 9, further comprising the step of selectively applying a negative pressure to a lumen in the catheter to change a curvature of the catheter.

16. The method according to claim 9, wherein the catheter has a plurality of lumens, and a plurality of resilient fiber cores having preformed curve shapes are inserted into respective lumens to create a desired curve shape in the catheter.

17. A catheter that can be deployed with a curved shape, comprising:
a resilient fiber core having a preformed shape; and
a polymer tubing having a lumen for receiving the resilient fiber core, said polymer tubing having a soft portion over at least part of its length that conforms to the preformed shape of the resilient fiber core.

18. The catheter according to claim 17, wherein the preformed shape of the resilient fiber core is a curved shape.

19. The catheter according to claim 17, wherein the catheter has a varying hardness over its length with a relatively hard portion that does not conform to the preformed shape of the resilient fiber core.

20. The catheter according to claim 17, wherein said polymer tubing comprises a second lumen for receiving a pressure to change a curvature of the catheter.

21. The catheter according to claim 17, wherein the polymer tubing comprises a plurality of lumens, and a plurality of resilient fiber cores having preformed curve shapes are inserted into respective lumens to create a desired curve shape in the catheter.
